(19) United States
(12) Patent Application Publication
(10) Pub. No.: US 2023/0000476 A1
(43) Pub. Date: Jan. 5, 2023

(54) IMPLANTABLE CLOSURE DEVICE TO SEAL PUNCTURES OF SPINAL CORD AND BRAIN MENINGES MEMBRANES AND PREVENT LEAKAGE OF CEREBROSPINAL FLUID

(71) Applicant: Deinde Medical Corp., Miramar, FL (US)

(72) Inventor: Robert C. Farnan, Fort Lauderdale, FL (US)

(21) Appl. No.: 17/941,114

(22) Filed: Sep. 9, 2022

Related U.S. Application Data
(63) Continuation of application No. PCT/US2021/021844, filed on Mar. 11, 2021.
(60) Provisional application No. 62/988,772, filed on Mar. 12, 2020.

(51) Int. Cl. A61B 17/00

(52) U.S. Cl. CPC A61B 17/0057; A61B 2017/00592; A61B 2017/00597; A61B 2017/00867

(57) ABSTRACT

An implantable closure device for sealing an opening through a biologic tissue membrane against leakage of biological fluid includes a fluid sealing plug configured to be positioned within the opening. One or more retainer sections are configured to secure the fluid sealing plug within the opening. The retainer sections include at least one of a distal retainer section coupled to a distal end of the fluid sealing plug and configured to be disposed distally on the biologic tissue membrane, or a proximal retainer section coupled to a proximal end of the fluid sealing plug and configured to be disposed proximally on the biologic tissue membrane.

### CROSS REFERENCE TO RELATED APPLICATION

This application is a continuation of PCT Application Serial No. PCT/2021/021844, filed Mar. 11, 2021, which claims priority to U.S. Provisional Patent Application Ser. No. 62/988,772 filed on Mar. 12, 2020, the disclosures of which are incorporated herein by reference in their entirety.

### FIELD OF THE INVENTION

The present systems, apparatuses, and methods lie in the field of implantable closure devices. The present disclosure relates to an implantable closure device to seal punctures or holes in Meninges membranes of the spinal cord and brain and prevent leakage of the cerebrospinal fluid (CSF) into the soft tissue space (i.e. fat, skin, muscle) that surround the Meninges membranes and CSF system. The disclosure also relates to methods for manufacture and use of the closure devices.

### BACKGROUND OF THE INVENTION

The Meninges are protective membranes that surround the brain and spinal cord. These membranes (the dura, arachnoid, and pia layers as shown in FIG. 1) encapsulate the CSF to form a conduit that surrounds the spinal cord and the cerebral ventricles. Typically, a needle is used to puncture through the skin and Meninges membranes to gain access to the CSF. When the needle is removed, the hole created does not consistently seal due to the inelastic properties of the membranes, causing CSF leakage into the soft tissue that surrounds these membranes, which is not clinically desirable.

Closure devices exist that are used in the vascular system, for example, access closure devices, PFO closure devices, and ASD closure devices. However, none relate to closure of the Meninges for preventing the leakage of CSF.

Thus, a need exists to overcome the problems with the prior art systems, designs, and processes as discussed above.

### SUMMARY OF THE INVENTION

The systems, apparatuses, and methods described provide an implantable closure device to seal punctures or holes in meninges membranes of the spinal cord and brain and prevent leakage of the cerebrospinal fluid and methods for manufacture and use of the closure devices that overcome the hereinafore-mentioned disadvantages of the heretofore-known devices and methods of this general type and that provide such features with a unique structure having high efficiency in leakage prevention.

To close the puncture, or access site, the closure device incorporates a superelastic structure having the ability to be deformed into a low-profile shape for delivery into the puncture site then to expand radially to a preformed shape to engage and close the puncture site. This Meninges closure device can also incorporate a porous polymer structure that promotes the healing of the membranes to further reinforce sealing of the puncture site. The delivery and deployment of the closure device is conducted using clinically acceptable visualization techniques (e.g. Fluoroscopic, Endoscope, Computed Tomography Scan).

As used herein, materials that are super-elastic or superelastic include those alloys that belong to the larger family of shape-memory alloys and materials that reversibly deform in an equivalent manner. When mechanically loaded, a superelastic alloy deforms reversibly to very high strains (up to 10%) by the creation of a stress-induced phase. When the load is removed, the new phase becomes unstable and the material regains its original shape. Use of superelastic in regards to materials include those materials and alloys where no change in temperature is needed for the alloy to recover its initial shape. Superelastic devices take advantage of their large, reversible deformation and include antennas, eyeglass frames, and biomedical devices including stents. Nickel titanium (or Nitinol®) is an example of an alloy exhibiting superelasticity.

With the foregoing and other objects in view, there is provided, a system for sealing punctures in Meninges membranes, comprising a hollow needle assembly comprising a hollow needle sheath assembly having a proximal end, a distal end, a sheath hub, and an outer diameter, and defining a sheath lumen extending from the proximal end to the distal end, and a needle having a needle hub and a shape substantially corresponding to the sheath lumen to traverse through the sheath lumen from the proximal end and out the distal end. The system includes an implantable, puncture-sealing, closure device comprising a set of first struts of a superelastic material that is expandable and contractable, the first struts having a steady state extended within a first strut plane, the first struts together defining a first center joint, a set of second struts of a superelastic material that is expandable and contractable, the second struts having a steady state extended within a second strut plane substantially parallel to the first strut plane, the second struts together defining a second center joint, and a plug segment. The plug segment is of a superelastic material that is expandable and contractable, defines a longitudinal axis between the first and second center joints, has a first end connected to the first center joint and a second end opposite the first end connected to the second center joint such that, in an uncompressed steady state, the first and second sets of struts are substantially parallel to one another, has a height between approximately 240 microns and approximately 300 microns, and has a steady state that, when uncompressed in a radial direction, is between 1.5 and 3 times the diameter of the needle sheath. The closure device is collapsible to fit and slide within the sheath lumen.

Generally, an implantable closure device for sealing an opening through a biologic tissue membrane against leakage of biological fluid is provided. The closure device includes a fluid sealing plug configured to be positioned at least partially within the opening through a biologic tissue membrane, and at least one retainer section configured to secure the fluid sealing plug at least partially within the opening. The retainer section or sections include a distal retainer section configured to be disposed distally on the biologic tissue membrane, and/or a proximal retainer section configured to be disposed proximally on the biologic tissue membrane. The fluid sealing plug is coupled to the distal retainer section at the distal end of the sealing plug, and/or the fluid sealing plug is coupled to the proximal retainer section at the proximal end of the sealing plug. The closure device has a longitudinal axis defined generally as a centerline of the liquid sealing plug passing generally from the distal end of the liquid sealing plug to the proximal end of the liquid sealing plug through the center of the liquid sealing plug.

In some embodiments, the fluid sealing plug may have a generally radial outer surface configured to engage a generally radial inner surface of the opening to provide an at least partially sealed interface between the generally radial outer surface of the fluid sealing plug and the generally radial inner surface of the opening. The fluid sealing plug may be reconfigurable between a low-profile delivery configuration and an implanted configuration. The fluid sealing plug may include superelastic wire.

In alternate or additional aspects, the distal retainer section includes a retainer structure including a plurality of expandable struts forming the distal retainer section, and the expandable struts may be arranged radially to the longitudinal axis of the closure device. The distal retainer section may include superelastic wire. The proximal retainer section may include a retainer structure including a plurality of expandable struts forming the proximal retainer section, and the expandable struts may be arranged radially to the longitudinal axis of the closure device. The proximal retainer section may include superelastic wire.

In some embodiments, the distal retainer section may be reconfigurable between a low-profile delivery configuration and an implanted configuration. In the implanted configuration, a diameter of the distal retainer section may be greater than a diameter of the fluid sealing plug. In the implanted configuration, the diameter of the distal retainer section may be about 1.25 to about 3.0 times a diameter of the opening and the diameter of the fluid sealing plug. In an implanted configuration, the distal retainer section may be disposed generally in a plane oriented substantially perpendicular to the longitudinal axis of the closure device. The proximal retainer may be reconfigurable between a low-profile delivery configuration and an implanted configuration. In the implanted configuration, a diameter of the proximal retainer section may be greater than a diameter of the fluid sealing plug. In the implanted configuration, the diameter of the proximal retainer section may be about 1.25 to about 3.0 times a diameter of the opening and the diameter of the fluid sealing plug. In an implanted configuration, the proximal retainer section is disposed generally in a plane oriented substantially perpendicular to the longitudinal axis of the closure device.

In alternate or additional aspects, the closure device may include a distal retainer section cover at least partially covering the distal retainer section. The distal retainer section cover may include a porous polymer material. The closure device may include a proximal retainer section cover at least partially covering the proximal retainer section. The proximal retainer section cover may include a porous polymer material.

In some embodiments, the closure device may include a porous polymer material within the fluid sealing plug. The porous polymer material may be coupled to one or both ends of the fluid sealing plug. The porous polymer material may include a plurality of fibers. The implantable closure device may include a marker configured to be detectable using a medical imaging technique.

In another embodiment, an implantable closure device for sealing an opening through a biologic tissue membrane against leakage of biological fluid is provided. The implantable closure device is reconfigurable between a low-profile delivery configuration and an implanted configuration. The closure device includes a fluid sealing plug including superelastic wire and configured to be positioned at least partially within the opening through a biologic tissue membrane. The closure device also includes a plurality of retainer sections including superelastic wire and configured to secure the fluid sealing plug at least partially within the opening. The retainer sections include a distal retainer section including a plurality of radially oriented expandable struts configured to be disposed distally on the biologic tissue membrane, and a proximal retainer section including a plurality of radially oriented expandable struts configured to be disposed proximally on the biologic tissue membrane. The fluid sealing plug is coupled to the distal retainer section at the distal end of the sealing plug. The fluid sealing plug is coupled to the proximal retainer section at the proximal end of the sealing plug. The closure device has a longitudinal axis defined generally as a centerline of the liquid sealing plug passing generally from the distal end of the liquid sealing plug to the proximal end of the liquid sealing plug through the center of the liquid sealing plug. The distal retainer expandable struts are arranged radially to the longitudinal axis of the closure device. The proximal retainer expandable struts are arranged radially to the longitudinal axis of the closure device.

In an alternate embodiment, a closure device delivery system including a closure device and a loading device is provided. The loading device includes a generally longitudinal lumen containing the closure device therein, the closure device being in a delivery configuration when contained in the loading device. The loading device may include a pusher assembly configured to deploy the closure device from the loading device. The pusher assembly may include a coupler releasably connected to the proximal end of the fluid sealing plug. The closure device may be configured to transition from the delivery configuration to the implanted configuration as it is deployed from the loading device. The closure device delivery system may include a delivery sheath assembly including a lumen extending therethrough. The loading device and the delivery sheath assembly may be configured to releasably couple together such that the lumen of the delivery sheath assembly is generally axially aligned with a lumen of the loading device. The closure device delivery system may include a needle assembly. The needle assembly and the delivery sheath assembly may be configured to releasably couple together such that at least a portion of the needle assembly extends through the lumen of the delivery sheath assembly.

A method of closing an opening through a biologic tissue membrane and sealing against leakage of biological fluid is provided. The method includes deploying a distal retainer portion of a closure device distal to a distal surface of a biologic tissue membrane through an opening through the biologic tissue membrane, deploying a fluid sealing plug coupled to the distal retainer portion of the closure device at least partially into the opening, and deploying a proximal retainer portion coupled to the fluid sealing plug of the closure device proximal to a proximal surface of the biologic tissue membrane.

In some embodiments, deploying the distal retainer portion of the closure device includes transitioning the distal retainer portion from a delivery configuration to an implanted configuration. Deploying the proximal retainer portion of the closure device may include transitioning the proximal retainer portion from a delivery configuration to an implanted configuration. Deploying the distal retainer portion of the closure device may include deploying the distal retainer portion distally spaced apart from the distal surface of the biologic tissue membrane, and prior to deploying the fluid sealing plug, withdrawing the closure device to place the distal retainer portion in contact with the distal surface of the biologic tissue membrane. The method may include advancing a generally tubular delivery sheath assembly through the opening and positioning a loading device on the delivery sheath assembly before deploying the distal retainer portion of the closure device. The loading device may include a lumen containing the closure device in a delivery configuration. Deploying the distal retainer portion of the closure device, deploying the fluid sealing plug, and deploying the proximal retainer portion may include deploying the closure device from the loading device and through the delivery sheath assembly.

In alternate or additional aspects, deploying the distal retainer portion of the closure device may include extending the distal retainer portion of the closure device from a distal tip of the delivery sheath assembly and transitioning the distal retainer portion from the delivery configuration to an implanted configuration. Deploying the proximal retainer portion of the closure device may include extending the proximal retainer portion of the closure device from a distal tip of the delivery sheath assembly and transitioning the proximal retainer portion from the delivery configuration to an implanted configuration. Deploying the closure device from the loading device may include advancing distally a pusher assembly, the pusher assembly being configured to push the closure device distally from the lumen of the loading device and through a lumen of the delivery sheath assembly. Deploying the proximal retainer portion of the closure device may include detaching the fluid sealing plug portion of the closure device from the pusher assembly.

In alternate or additional aspects, the method may include assessing at least one of a position of the closure device and an efficacy of the closure device, prior to detaching the fluid sealing plug portion from the pusher assembly. Deploying the fluid sealing plug may include withdrawing the delivery sheath assembly from the opening. Advancing the generally tubular delivery sheath assembly through the opening through the biologic tissue membrane may include advancing together the delivery sheath assembly and a needle assembly extending through the lumen of the delivery sheath assembly at least until a tip of the delivery sheath assembly has penetrated the biologic tissue membrane. The method may include removing the needle assembly from the delivery sheath assembly before positioning the loading device on the delivery sheath assembly.

Although the systems, apparatuses, and methods are illustrated and described herein as embodied in an implantable closure device to seal punctures or holes in meninges membranes of the spinal cord and brain and prevent leakage of the cerebrospinal fluid and methods for manufacture and use of the closure devices, it is, nevertheless, not intended to be limited to the details shown because various modifications and structural changes may be made therein without departing from the spirit of the invention and within the scope and range of equivalents of the claims. Additionally, well-known elements of exemplary embodiments will not be described in detail or will be omitted so as not to obscure the relevant details of the systems, apparatuses, and methods.

Additional advantages and other features characteristic of the systems, apparatuses, and methods will be set forth in the detailed description that follows and may be apparent from the detailed description or may be learned by practice of exemplary embodiments. Still other advantages of the systems, apparatuses, and methods may be realized by any of the instrumentalities, methods, or combinations particularly pointed out in the claims.

Other features that are considered as characteristic for the systems, apparatuses, and methods are set forth in the appended claims. As required, detailed embodiments of the systems, apparatuses, and methods are disclosed herein; however, it is to be understood that the disclosed embodiments are merely exemplary of the systems, apparatuses, and methods, which can be embodied in various forms.

Therefore, specific structural and functional details disclosed herein are not to be interpreted as limiting, but merely as a basis for the claims and as a representative basis for teaching one of ordinary skill in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the systems, apparatuses, and methods of the invention that are regarded as novel, it is believed that the systems, apparatuses, and methods will be better understood from a consideration of the following description in conjunction with the drawing figures, in which like reference numerals are carried forward.

### BRIEF DESCRIPTION OF THE DRAWINGS

The accompanying figures, where like reference numerals refer to identical or functionally similar elements throughout the separate views, which are not true to scale, and which, together with the detailed description below, are incorporated in and form part of the specification, serve to illustrate further various embodiments and to explain various principles and advantages all in accordance with the systems, apparatuses, and methods. Advantages of embodiments of the systems, apparatuses, and methods will be apparent from the following detailed description of the exemplary embodiments thereof, which description should be considered in conjunction with the accompanying drawings.

FIG. 1 is a cross-sectional view of a portion of protective Meninges membranes that surround the brain and the spinal cord.

FIG. 2 is a perspective view of an exemplary embodiment of a superelastic Meninges closure device.

FIG. 3 is a cross-sectional view of the closure device of FIG. 2 sealing a punctured Meninges membrane.

FIG. 4 is a cross-sectional view of an exemplary embodiment of a needle and sheath puncture device inserted through the Meninges membrane into a CSF space.

FIG. 5 is a cross-sectional view of a closure device prepared for deployment in a loading device.

FIG. 6 is a cross-sectional view of an exemplary embodiment of the loading device/closure device of FIG. 5 engaged with the sheath puncture device of FIG. 4.

FIG. 7 is a cross-sectional view of the closure device of FIG. 5 advanced into the sheath assembly of FIG. 4.

FIG. 8 is a cross-sectional view of the closure device and sheath assembly of FIG. 7 with the closure device advanced further into the sheath assembly with distal struts extended away from the distal side of the Meninges in the CSF space.

FIG. 9 is a cross-sectional view of the closure device and sheath assembly of FIG. 8 with the distal struts extended and resting against the Pia membrane in the CSF space.

FIG. 10 is a cross-sectional view of the closure device and sheath assembly of FIG. 9 with the proximal struts of the closure device extended against the Dura membrane and capturing Meninges layers therebetween the struts.

FIG. 11 is a cross-sectional view of the closure device and sheath assembly of FIG. 10 fully deployed and the needle and sheath puncture device removed from the closure device and sheath assembly and the Meninges.

FIG. 12 is a perspective view of another exemplary embodiment of a superelastic Meninges closure device with struts on a single side.

FIG. 13 is a perspective view of another exemplary embodiment of a superelastic Meninges closure device with covered struts on a single side and a plug.

FIG. 14 is a side view of another exemplary embodiment of a superelastic Meninges closure device with struts and filled with porous fibers.

FIG. 15 is a perspective view of the Meninges closure device of FIG. 14 filled with porous fibers and constrained but with a loading device removed.

FIG. 16 is a cross-sectional view of another exemplary embodiment of a superelastic Meninges closure device with a porous, knitted, woven disc around struts and a radiopaque marker with internal threads.

FIG. 17 is a top plan view of the Meninges closure device of FIG. 16.

FIG. 18 is a side elevational view of an exemplary embodiment of a control wire for delivery of a Meninges closure device.

### DETAILED DESCRIPTION OF THE EMBODIMENTS

As required, detailed embodiments of the systems, apparatuses, and methods are disclosed herein; however, it is to be understood that the disclosed embodiments are merely exemplary of the systems, apparatuses, and methods, which can be embodied in various forms. Therefore, specific structural and functional details disclosed herein are not to be interpreted as limiting, but merely as a basis for the claims and as a representative basis for teaching one skilled in the art to variously employ the systems, apparatuses, and methods in virtually any appropriately detailed structure. Further, the terms and phrases used herein are not intended to be limiting; but rather, to provide an understandable description of the systems, apparatuses, and methods. While the specification concludes with claims defining the features of the systems, apparatuses, and methods that are regarded as novel, it is believed that the systems, apparatuses, and methods will be better understood from a consideration of the following description in conjunction with the drawing figures, in which like reference numerals are carried forward.

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Alternate embodiments may be devised without departing from the spirit or the scope of the invention. Additionally, well-known elements of exemplary embodiments of the systems, apparatuses, and methods will not be described in detail or will be omitted so as not to obscure the relevant details of the systems, apparatuses, and methods.

Before the systems, apparatuses, and methods are disclosed and described, it is to be understood that the terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact (e.g., directly coupled). However, “coupled” may also mean that two or more elements are not in direct contact with each other, but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in turn, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Herein various embodiments of the systems, apparatuses, and methods are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

### DESCRIPTION OF COMPONENTS

1. Superelastic Closure Device
2. Expandable Struts
2a. Distal Expandable Struts
2b. Proximal Expandable Struts
3. Expandable Plug Segment
4. Distal Wire Joint
5. Release Housing/Proximal Wire Joint
6. Pia Membrane
7. Arachnoid Membrane
8. Dura Membrane
9. Cerebrospinal Fluid (CSF)
10. Soft Tissue Matter
11. Needle Assembly
12. Needle Tip
13. Needle Hub
14. Delivery Sheath Assembly
15. Sheath Tip
16. Sheath Hub
17. Needle/Sheath Interface
18. Puncture Channel
19. Constrained Closure Device
20a. Distal Constrained Strut
20b. Proximal Constrained Strut
21. Constrained Expandable Plug Segment
22. Pusher Assembly
23. Release Wire
24. Pusher Stop
25. Loading Device
26. Sheath/Loading Device Interface
27. Closure Device Release Mechanism
28. Remaining Wound in Soft Tissue Matter
30. Closure Device—Single Sided Struts
40. Closure Device—Covered Struts and Plug
41. Covered Expandable Struts
42. Covered Expandable Plug Segment
50. Closure Device
51. Porous Fiber(s)
52. Covered Expandable Plug Segment
53. Brain
54. Closure Device Axis
55. Sheath Lumen
56. Loading Device Lumen
57. Loading Device Distal End
58. Needle Sheath Assembly Proximal End
59. Needle Sheath Assembly Outer Diameter
60. Closure Device—Covered Struts
61. Expandable Strut Cover
62. Radiopaque Marker
63. Internal Threads

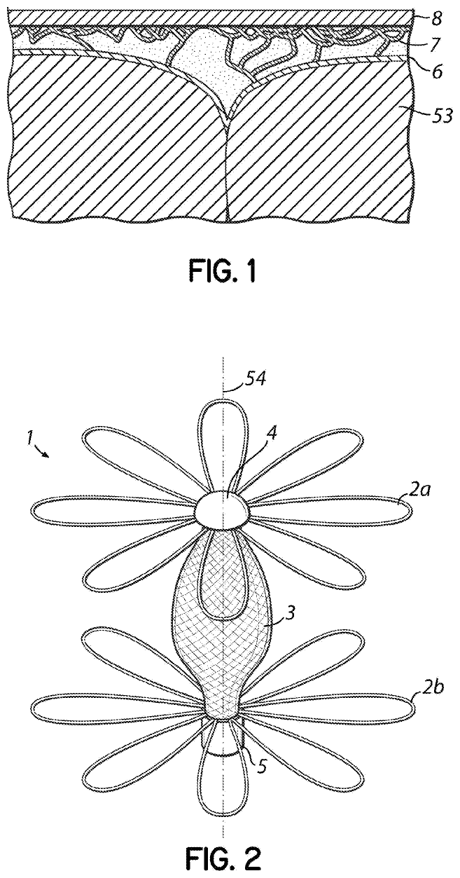

Described now are exemplary embodiments. FIG. 1 is a cross-sectional view of a portion of protective Meninges membranes, the Pia membrane 6, Arachnoid membrane 7, and Dura membrane 8, that surround the brain 53 and the spinal cord (not shown). Referring now to the figures of the drawings in detail and first, particularly to FIG. 2, there is shown a first exemplary embodiment of a Meninges superelastic closure device 1 in its pre-formed shape. This closure device incorporates two sets (distal and proximal ends of the device) of superelastic struts 2a, 2b. Extending between these struts is a superelastic plug segment 3. The struts 2a, 2b are used for securing the closure device between the inner and outer surfaces of the Meninges while the plug segment 3 is used to seal the puncture through the three membrane layers. Strut lengths may range from 1.25× to 3× the diameter of the Meninges hole. The entire device is formed using superelastic wire (e.g., a nickel-titanium alloy such as Nitinol) configured to form these features. The size and number of wires used to create the device are configured to allow deployment through devices/instruments that are used to gain access to the cerebrospinal fluid space. The distal end of the wires are joined by a welding or bonding process (e.g., laser welding, adhesive encapsulation, polymer or metallic feature that accepts the individual wires) to form the distal wire joint 4 that provides a radiused feature to minimize trauma to any adjacent anatomical feature near or opposing the puncture site. The proximal end of the wires is also configured to be assembled and joined into a release housing 5 feature using similar methods as the distal wire joint 4. The release housing 5 allows for a controlled delivery and deployment of the closure device 1. The number of struts 2a, 2b on both the distal and proximal ends of the device may vary from two struts up to a maximum of thirty-two struts depending upon the size of hole to be closed and size of the delivery device. The sizes of wires used to form the device may also vary from 0.0127 mm (0.0005″) in diameter to 0.127 mm (0.0050″) in diameter to allow for the tailoring of the retention force to dislodge the device.

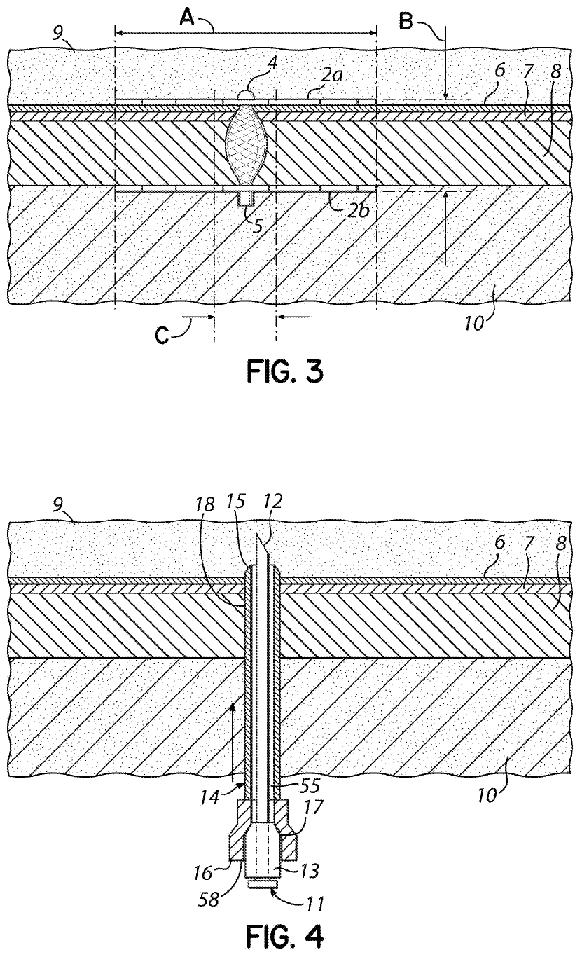

FIG. 3 shows a cross-section of the closure device 1 in its deployed state across the three layers of the Meninges. As shown, the distal set of struts 2a and distal wire joint 4 reside in the CSF 9 space with the distal struts 2a in contact with the Pia mater 6 of the Meninges. The plug segment 3 then spans between the three layers of the Meninges and expands radially to create the seal through the individual membrane layers, the Pia mater 6, the Arachnoid mater 7, and the Dura mater 8. It is desirable to have the plug segment 3 expand to a minimum of 0.75× to account for any retraction of the membranes as the needle/access device is removed. Maximum expansion of the plug segment 3 is determined experimentally by the onset of a tear in the Meninges layers (estimated to be approximately 2× the size of the resultant puncture hole). Also shown are the relative positioning of the proximal features of the closure device with the proximal struts 2b in contact with the Dura mater 8 and the release housing 5 extending into the soft tissue matter 10. The struts 2a, 2b may also be formed to allow for additional deflection towards the Meninges membranes for further securement of the closure device to the membrane layers. This deflection may vary from a line drawn perpendicular to the axis of the closure device 54 to an inward angle of up to approximately 15 degrees per strut.

The remaining figures are used to demonstrate the method and accessory devices that are used to deploy the closure device. As mentioned above, deployment of the device is conducted using clinically acceptable visualization techniques and the closure device and delivery accessories include materials or geometric features that aid in the preferred visualization technique.

FIG. 4 illustrates the use of a needle assembly 11 inserted into the lumen 55 of the closure device's delivery sheath assembly 14. The inner diameter of the sheath assembly 14 is designed to fit closely to the outer diameter of the needle assembly 11 while minimizing the gap in the annulus space between the devices (approximately 0.0254 mm (0.001″) to 0.127 mm (0.005″)). This fit is important to ensure minimal entrapment of the membrane layers during advancement while allowing for easy removal of the needle assembly 11 from the sheath assembly 14. As shown, both assemblies are advanced together through the soft tissue and Meninges membranes until the needle tip 12 and the sheath tip 15 reside within the CSF 9 space. The relative distance between the needle tip 12 and the sheath tip 15 is controlled by the length of the sheath assembly 14 and the engagement of a sheath hub 16 and a needle hub 13 at the surface where the two hubs 16, 13 are in contact, the needle/sheath interface 17. It is desirable to position the sheath tip 15 just proximal to the lumen on the needle assembly 11. It is noted that the leading edge(s) of the sheath tip 15 should be rounded (or tapered) to facilitate insertion through the soft tissue and Meninges membranes.

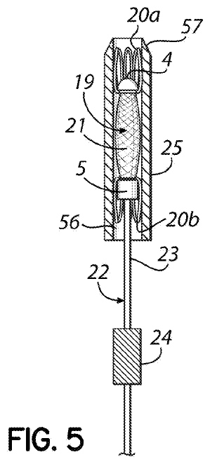

FIG. 5 depicts a constrained closure device 19 in preparation for insertion into the sheath assembly 14 (not shown). To constrain the constrained closure device 19, a loading device 25 is used. This loading device has a lumen 56 extending through its body with a size that is the same as, or minimally smaller than, the lumen 55 of the sheath assembly 14. The superelastic properties of the closure device 1 are now appreciated with the deformation of the distal and proximal constrained struts 20a, 20b configured to be opposed within the loading device 25. This positioning of the struts 20a, 20b is important to ensure that each set of struts 20a, 20b will correctly engage the Meninges Pia membrane 6 and Dura membrane 8, respectively, during deployment of the constrained closure device 19. Also, spanning the distance between the two sets of constrained struts 20a, 20b resides the feature of the constrained closure device 19 as a constrained expandable plug segment 21. The superelastic properties of the material used to construct the constrained closure device 19 is evident by the deformation of the features that comprise the device into an easily constrained state within the loading device 25. FIG. 5 additionally discloses a pusher assembly 22, which is configured to engage the constrained closure device 19 at the release housing 5 and control movement of the closure device 19 through each phase of deployment. The pusher assembly 22 includes a release wire 23 and a pusher stop 24. The release wire 23 is generally comprised of a metallic structure (or rigid polymer structure) that allows the constrained closure device 19 to be advanced and retracted with approximately 1:1 movement between the physician's hand and the tip (the most distal end of the distal set of the struts 20a) of the closure device 19. The pusher stop 24 may present as a removable component or be configured to include the ability to slide on the release wire 23, or both. It is important to note that the pusher stop 24 must remain in its predetermined assembled position until it is deliberately or intentionally removed/repositioned by the physician.

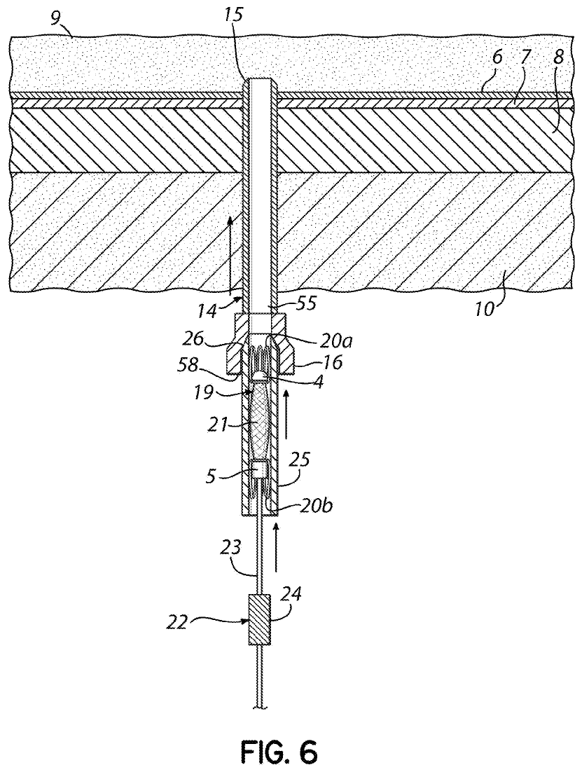

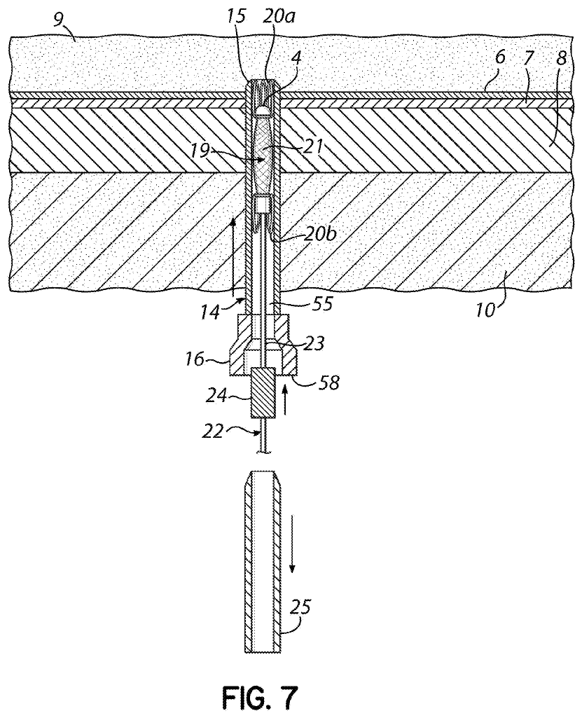

FIG. 6 shows the needle assembly 11 (not shown) removed from the sheath assembly 14 and engages distal edge features of the loading device distal end 57 into the cavity of the sheath hub 16 until the sheath/loading device interface 26 is created. It is important to maintain the position of the sheath tip 15 within the CSF 9 space while the sheath assembly 14 and loading device 25 are assembled. It is also important to note that, when creating this sheath/loading device assembly, all features of the constrained closure device 19 must remain housed within the lumen of the loading device 25. FIG. 6 also depicts initiation of deployment of the constrained closure device 19. To begin, the sheath assembly 14 and loading device 25 relative positioning must be held constant in their assembled positions, again with the sheath tip 15 in the CSF. The pusher assembly 22 is then slowly advanced to initiate the movement of the constrained closure device 19 towards the lumen 55 of the sheath assembly 14. FIG. 7 shows the constrained closure device 19 advanced entirely into the lumen 55 of the sheath assembly 14 by advancement of only the pusher assembly 22. With the constrained closure device 19 now residing within the sheath assembly 14, the loading device 25 may now be removed from the sheath hub 16 and set aside. When advancing the constrained closure device 19 into the sheath assembly 14, it is important to maintain the position of the sheath tip 15 within the CSF 9 as shown.

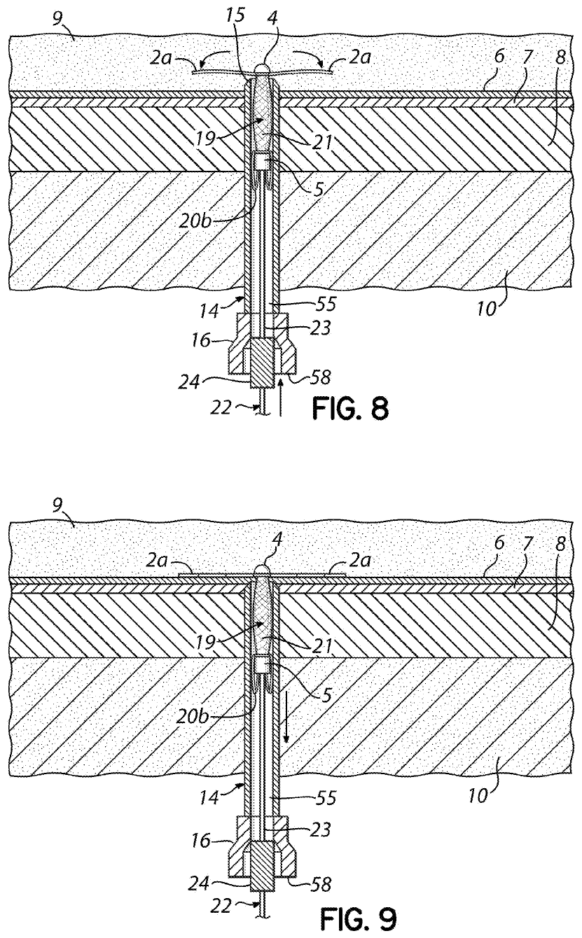

A first step in the deployment of the constrained closure device 19 is illustrated in FIG. 8. As the pusher assembly 22 is slowly advanced, the extension of the distal expandable struts 2a open as they exit the sheath tip 15 and return to their originally pre-formed position. Once the distal set of struts 2a have expanded, their relative positioning to the Pia membrane 6 is determined by the distance the sheath tip 15 extends into the CSF 9 and original shape of the distal struts 2a. To ensure that only the distal strut section 2a of the constrained closure device 19 is deployed, the pusher stop 24 is pre-positioned on the release wire 23 so that the pusher stop 24 engages the sheath hub 16, thereby preventing further advancement of the remaining features of the constrained closure device 19.

FIG. 9 shows the distal struts 2a in contact with the Pia membrane 6. This positioning is accomplished by holding the relative positioning of the sheath assembly 14 and the pusher assembly 22 constant, then slowly retracting both assemblies 14, 22 until resistance of the distal struts 2a engaging the Pia membrane 6 is felt (and observed).

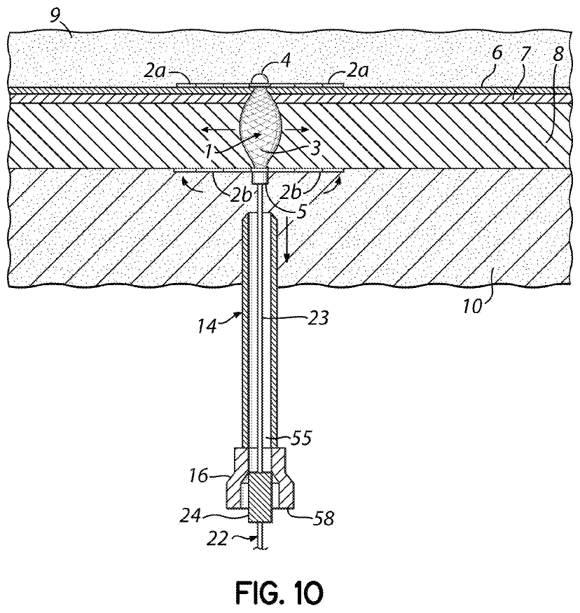

FIG. 10 illustrates deployment of the expandable plug segment 3 and the proximal expandable struts 2b. Prior to deployment, the pusher stop 24 is either removed from the pusher assembly 22 or repositioned to a pre-determined location along the release wire 23. Once the pusher stop 24 has been moved to an appropriate position, the delivery sheath assembly 14 may be slowly retracted, while holding stable the position of the pusher assembly 22 until the expandable plug segment 3 and proximal set of expandable struts 2b have exited the sheath assembly 14. With the sheath assembly 14 removed, the plug segment 3 occludes the puncture hole and compresses outwardly on the Meninges membranes to seal the puncture hole while the proximal struts 2b engage the Dura membrane 8 and create compression on the membrane layers to stabilize the closure device 1. The closure device 1 may now be monitored for its efficacy of closing the puncture hole. Additionally, if any/or all, of the features of the closure device 1 are not positioned correctly, the closure device 1 may be recaptured back into the sheath assembly 14 by simply pulling on the pusher assembly 22, and then safely removing the sheath assembly 14 and the pusher assembly 22 from the patient.

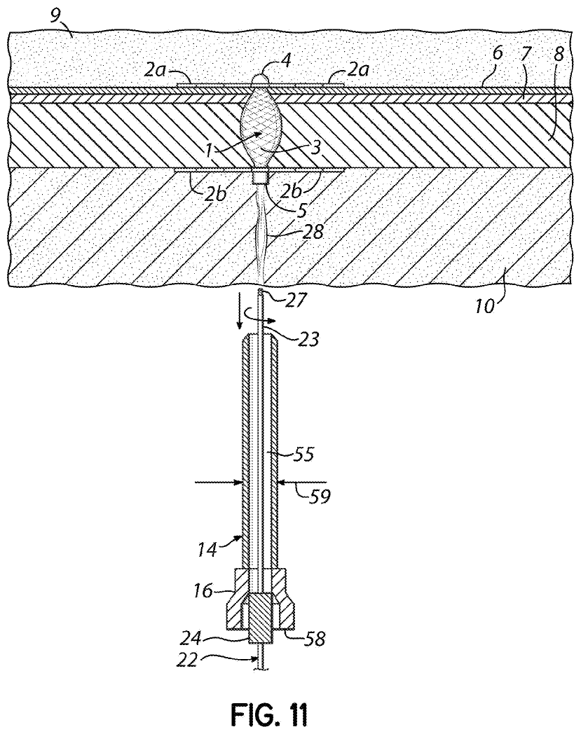

Final release of the superelastic closure device 1 is shown in FIG. 11. The closure device 1 is separated by activation of the closure device release mechanism 27 shown in FIGS. 11 and 18. Also referring to FIGS. 16 and 18, for purposes of this illustration, threaded mating features, internal threads 63 of the release housing 5 and closure device release mechanism 27 of the release wire 23, are used to demonstrate a separation of the closure device 1 from the release wire 23. By simply rotating the release wire 23, the pusher assembly 22 detaches from the closure device 1 in this configuration. The release wire 23 may also be fabricated using superelastic material and incorporate a feature that interfaces with the release housing 5 using a pre-formed shape. As the release wire 23 is retracted, the pre-formed wire shape deforms and releases the closure device 1. The sheath assembly 14 and the pusher assembly 22 may now be removed from the patient, leaving a small wound 28 in the soft tissue matter 10, which has elastic properties and will seal upon itself.

In an exemplary embodiment of the system for sealing punctures in Meniscus membranes, the system includes the hollow needle assembly 11 and the implantable, puncture-sealing, closure device 1. The hollow needle sheath assembly 14 has a proximal end 58, a sheath tip 15 at the distal end, a sheath hub 16, and an outer diameter 59, and defines a sheath lumen 55 extending from the proximal end 58 to the sheath tip 15 at the distal end. The sheath assembly 14 also has a needle assembly 11 having a needle hub 13 and a shape substantially corresponding to the sheath lumen 55 to traverse through the sheath lumen 55 from the proximal end 58 and out the sheath tip 15 at the distal end. The closure device 1 comprises distal struts 2a, proximal struts 2b, and a plug segment 3. The set of distal struts 2a is of a superelastic material that is expandable and contractable. The distal struts 2a have a steady state extended within a first strut plane, the distal struts 2a together defining a distal center joint 4. The set of proximal struts 2b is of a superelastic material that is expandable and contractable. The proximal struts 2b have a steady state extended within a second strut plane substantially parallel to the first strut plane. The proximal struts 2b together defining a release housing/proximal center joint 5. The plug segment 3 is of a superelastic material that is expandable and contractable. The plug segment 3 defines a longitudinal axis 54 between the distal and proximal center joints 4, 5, has a distal end connected to the distal center joint 4 and a proximal end opposite the distal end connected to the release housing/proximal center joint 5 such that, in an uncompressed steady state, the distal and proximal sets of struts 2a, 2b are substantially parallel to one another, has a height between approximately 240 microns and approximately 300 microns, and has a steady state that, when uncompressed in a radial direction, is between 1.5 and 3 times the diameter of the needle sheath 14. The closure device 1 is collapsible to fit and slide within the sheath lumen 55.

FIGS. 12, 13, 14, and 15 illustrate additional exemplary embodiments of various closure device configurations that are also viable for the closing of puncture holes in the Meninges membranes.

FIG. 12 discloses a closure device 30 with only one set of struts 2b. It is noted that the struts 2a or 2b may reside on either the distal or proximal ends of the plug 3 with identical positioning of the distal wire joint 4 and release housing/proximal center joint 5 remaining in their respective locations and configurations as described above.

FIG. 13 further expands on the options for configuring the closure device 40. In FIG. 13, the expandable struts 2a may be covered with a porous polymer structure to create covered expandable struts 41. FIG. 13 is only one exemplary illustration of how these covered struts 41 may be configured. It is apparent that the closure device may have the covered struts 41 configured as described above by having the covered struts 41 on either the distal, the proximal, or both ends of the expandable plug 42. It is also noted that the closure device 40 may include one covered set of struts 41 and one uncovered set of struts 2a or 2b

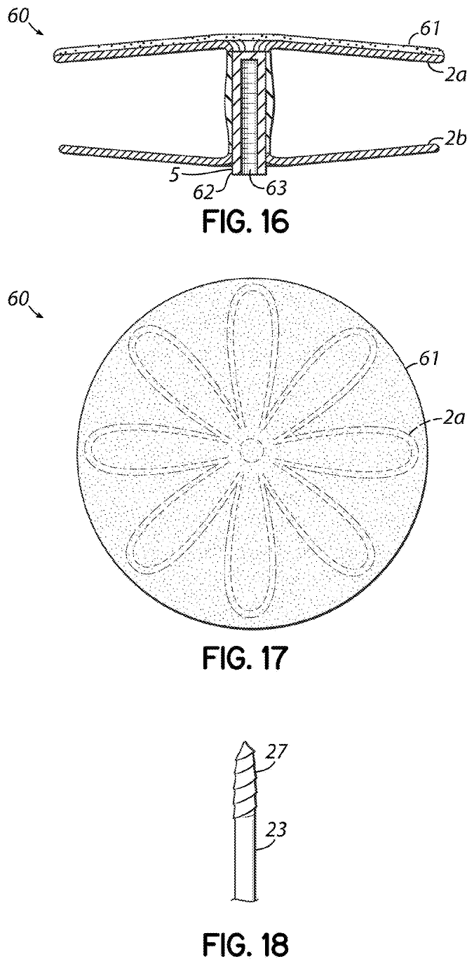

FIGS. 16 and 17 is another exemplary illustration of a closure device 60 with covered expandable struts 2a. In this exemplary illustration a cover 61 is generally in the shape of a disc covering the expandable struts 2a. FIGS. 16 and 17 is only one exemplary illustration of how the cover 61 may be configured. It is also noted that the closure device 60 may include one covered set of struts 2a or 2b and one uncovered set of struts 2a or 2b or both sets of struts 2a and 2b may be covered. Referring to FIG. 16, the illustrative closure device 60 may include a radiopaque marker 62. Referring to FIG. 16, the illustrative closure device 60 may include internal threads 63 as a means to engage the closure device release mechanism 27 of the release wire 23.

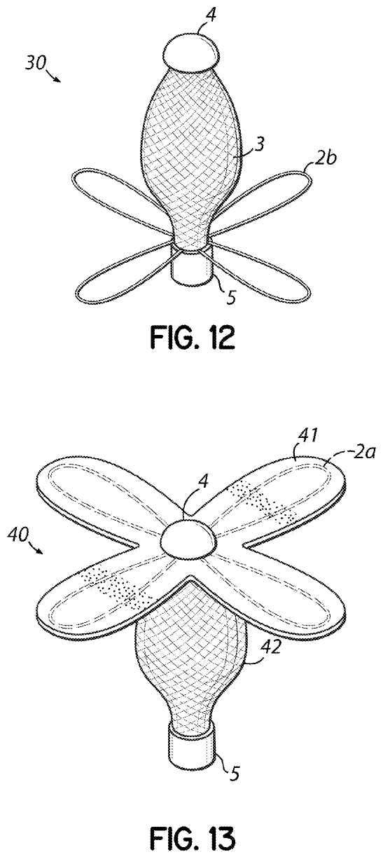

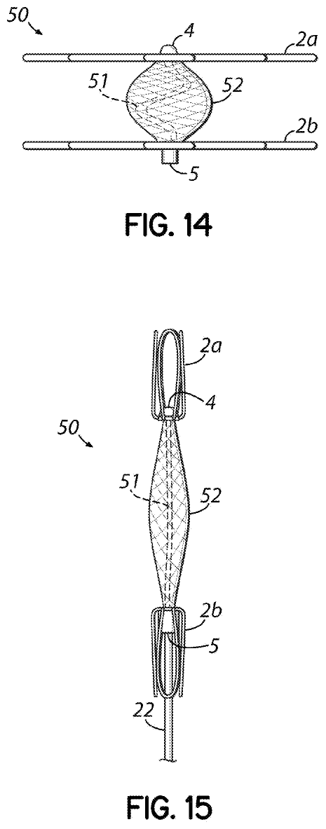

It is also conceivable that the closure device 50 includes porous polymer fibers that extend through and are contained within the expandable plug segment 52 of the closure device 50. FIGS. 14 and 15 illustrate an exemplary embodiment of a closure device 50 filled with porous fibers 51. These fibers may be secured at both ends of the expandable plug segment 52 where the distal wire joint 4 and the proximal wire joint/release housing 5 reside. FIG. 15 illustrates an exemplary extension of the fibers 51 when the closure device 50 and the expandable plug segment 52 are constrained within the loading device 25 (not shown for clarity) or the sheath assembly 14 (also not shown for clarity).

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The foregoing description and accompanying drawings illustrate the principles, exemplary embodiments, and modes of operation of the systems, apparatuses, and methods. However, the systems, apparatuses, and methods should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the systems, apparatuses, and methods as defined by the following claims.

What is claimed is:

1. An implantable closure device for sealing an opening through a biologic tissue membrane against leakage of biological fluid, the closure device comprising:
a fluid sealing plug configured to be positioned at least partially within the opening through the biologic tissue membrane; and
one or more retainer sections configured to secure the fluid sealing plug at least partially within the opening, the one or more retainer sections comprising at least one of:
a distal retainer section coupled to a distal end of the fluid sealing plug and configured to be disposed distally on the biologic tissue membrane; or
a proximal retainer section coupled to a proximal end of the fluid sealing plug and configured to be disposed proximally on the biologic tissue membrane;
wherein the device has a longitudinal axis defined generally as a centerline of the fluid sealing plug passing generally from the distal end of the fluid sealing plug to the proximal end of the fluid sealing plug through the center of the fluid sealing plug.

2. The device of claim 1, wherein a generally radial outer surface of the fluid sealing plug is configured to engage a generally radial inner surface of the opening to provide an at least partially sealed interface between the generally radial outer surface of the fluid sealing plug and the generally radial inner surface of the opening.

3. The device of claim 1, wherein the fluid sealing plug is reconfigurable between a low-profile delivery configuration and an implanted configuration.

4. The device of claim 1, wherein the fluid sealing plug comprises superelastic wire.

5. The device of claim 1, wherein the device includes the distal retainer section, and the distal retainer section comprises a retainer structure comprising a plurality of expandable struts forming the distal retainer section, and the expandable struts are arranged radially to the longitudinal axis of the closure device.

6. The device of claim 1, wherein the device includes the distal retainer section, and the distal retainer section comprises superelastic wire.

7. The device of claim 1, wherein the device includes the proximal retainer section, and the proximal retainer section comprises a retainer structure comprising a plurality of expandable struts forming the proximal retainer section, and the expandable struts are arranged radially to the longitudinal axis of the closure device.

8. The device of claim 1, wherein the device includes the proximal retainer section, and the proximal retainer section comprises superelastic wire.

9. The device of claim 1, wherein the device includes the distal retainer section, and the distal retainer section is reconfigurable between a low-profile delivery configuration and an implanted configuration, and in the implanted configuration, a diameter of the distal retainer section is greater than a diameter of the fluid sealing plug.

10. The device of claim 1, wherein the device includes the proximal retainer section, and the proximal retainer is reconfigurable between a low-profile delivery configuration and an implanted configuration, and in the implanted configuration, a diameter of the proximal retainer section is greater than a diameter of the fluid sealing plug.

11. The device of claim 1, wherein the device includes the distal retainer section, and the device further comprises a distal retainer section cover covering at least a section of the distal retainer section.

12. The device of claim 1, wherein the device includes the proximal retainer section, and the device further comprises a proximal retainer section cover covering at least a section of the proximal retainer section.

13. The device of claim 1, further comprising a porous polymer material within the fluid sealing plug.

14. The device of claim 13, wherein the porous polymer material comprises a plurality of fibers.

15. The device of claim 1, further comprising a marker configured to be detectable using a medical imaging technique.

16. A closure device delivery system comprising:
the closure device of claim 1; and
a loading device comprising a generally longitudinal lumen containing the closure device therein, the closure device being in a delivery configuration when contained in the loading device; and
a pusher assembly configured to deploy the closure device from the loading device;
wherein the closure device is configured to transition from the delivery configuration to the implanted configuration as it is deployed from the loading device.

17. The system of claim 16, wherein the pusher assembly comprises a coupler releasably connected to the proximal end of the fluid sealing plug.

18. The system of claim 16, further comprising a delivery sheath assembly comprising a lumen extending therethrough;
wherein the loading device and the delivery sheath assembly are configured to releasably couple together such that the lumen of the delivery sheath assembly is generally axially aligned with a lumen of the loading device.

19. The system of claim 18,
further comprising a needle assembly;
wherein the needle assembly and the delivery sheath assembly are configured to releasably couple together such that at least a section of the needle assembly extends through the lumen of the delivery sheath assembly.

20. A method of closing an opening through a biologic tissue membrane and sealing against leakage of biological fluid, the method comprising:
deploying a distal retainer section of a closure device distal to a distal surface of a biologic tissue membrane through an opening through the biologic tissue membrane;
deploying a fluid sealing plug coupled to the distal retainer section of the closure device at least partially into the opening; and
deploying a proximal retainer section coupled to the fluid sealing plug of the closure device proximal to a proximal surface of the biologic tissue membrane,
wherein deploying the distal retainer section of the closure device comprises transitioning the distal retainer section from a delivery configuration to an implanted configuration, and deploying the proximal retainer section of the closure device comprises transitioning the proximal retainer section from a delivery configuration to an implanted configuration.

21. The method of claim 20,
wherein deploying the distal retainer section of the closure device comprises deploying the distal retainer section distally spaced apart from the distal surface of the biologic tissue membrane; and
the method further comprising, prior to deploying the fluid sealing plug, withdrawing the closure device to place the distal retainer section in contact with the distal surface of the biologic tissue membrane.

22. The method of claim 20,
further comprising, before deploying the distal retainer section of the closure device, advancing a generally tubular delivery sheath assembly through the opening, and positioning a loading device on the delivery sheath assembly, the loading device comprising a lumen containing the closure device in a delivery configuration; and
wherein deploying the distal retainer section of the closure device, deploying the fluid sealing plug, and deploying the proximal retainer section comprise deploying the closure device from the loading device and through the delivery sheath assembly.

23. The method of claim 22, wherein deploying the distal retainer section of the closure device comprises extending the distal retainer section of the closure device from a distal tip of the delivery sheath assembly and transitioning the distal retainer section from the delivery configuration to an implanted configuration.

24. The method of claim 22, wherein deploying the closure device from the loading device comprises advancing distally a pusher assembly and detaching the fluid sealing plug section of the closure device from the pusher assembly, the pusher assembly being configured to push the closure device distally from the lumen of the loading device and through a lumen of the delivery sheath assembly.
